(19) United States
(12) Patent Application Publication
(10) Pub. No.: US 2004/0228829 A1
(43) Pub. Date: Nov. 18, 2004

(54) Plasma detoxification system and methods of use

(76) Inventors: Craig P. Roberts, Carlsbad, CA (US); Lon H. Stone, Dana Point, CA (US)

Correspondence Address: PRESTON GATES & ELLIS LLP, 1900 MAIN STREET, SUITE 600, IRVINE, CA, 92614-7319, US

(21) Appl. No.: 10/785,215

(22) Filed: Feb. 23, 2004

Related U.S. Application Data
(60) Provisional application No. 60/453,558, filed on Mar. 11, 2003.

(51) Int. Cl. A61K 35/78; A61K 31/74

(52) U.S. Cl. 424/78.1; 424/725.1; 604/500

(57) ABSTRACT

A device for removing toxins from the blood of patients suffering from acute liver failure, acute-on-chronic liver failure and sepsis is provided. The toxin removal device disclosed herein comprises activated charcoal and at least one non-ionic resin for use in an extracorporeal circuit that minimizes electrolyte and protein depletion from the treated plasma. Also provided are associated methods for treating patients suffering from acute liver failure, acute-on-chronic liver failure and sepsis using the disclosed toxin removal device.

### RELATED APPLICATIONS

The present application claims priority to U.S. Provisional Patent Application Ser. No. 60/453,558 filed Mar. 11, 2003.

### FIELD OF THE INVENTION

The present invention relates to devices and associated methods for plasma detoxification. Specifically, the present invention relates to an extracorporeal device that utilizes non-ionic resins and activated charcoal, either individually or in combination, to detoxify plasma from patients suffering from acute liver failure and/or sepsis.

### BACKGROUND OF THE INVENTION

The mammalian liver is essential for maintaining health and vitality. It is the primary organ responsible for eliminating toxins in the bloodstream, contributes to maintaining proper glucose levels in the blood; helps metabolize fats and synthesizes essential proteins such as albumin and blood clotting factors. Moreover, the liver plays an essential role in the endocrine and immune systems.

When the liver is damaged its ability to perform these and other vital functions is impaired resulting is a rapid decline in health. Most importantly, a damaged liver's ability to eliminate toxins from the blood stream may be temporarily overwhelmed which further exacerbates the problem. Liver failure requires immediate hospitalization because the toxin accumulation may rapidly lead to multi-organ failure including the brain (hepatic encephalopathy), the kidneys (leading to decreased urine output or kidney failure, which results in problems with fluid and electrolyte balance), the lungs (leading to conditions ranging from breathing difficulty to respiratory failure) and the cardiovascular system (leading to low blood pressure and sometimes complete cardiovascular collapse).

Liver disease may progress very slowly, sometimes over decades (chronic liver disease), or can develop rapidly (acute liver disease). “Chronic” liver disease progresses over an extended period and often leads to liver failure. Many chronic patients experience periodic acute events in which there is a sudden worsening of the disease. These events are referred to as “acute-on-chronic” events. Liver failure that develops quickly in a patient with a previously normal liver and is accompanied by encephalopathy is known as fulminant hepatic failure or FHF (Anand A. C., Nightingale P., Neuberger J. M., Early indicators of prognosis in fulminant hepatic failure: an assessment of the King's criteria. J Hepatology 1997; 26: 62-68).

The end result of chronic liver disease is cirrhosis or scarring of the liver, which distorts the structure of the liver and blocks blood flow through the liver. Patients can have a considerable amount of cirrhosis and still feel normal or experience minimal symptoms such as weakness, swelling, or fluid in the abdomen. However, when these patients experience medical complications on top of cirrhosis such as esophageal bleeding, infection, or poor nutrition, liver cells stop functioning, toxin levels increase, and liver cells die. This acute-on-chronic condition is referred to as decompensated cirrhosis.

Acute liver failure (ALF) most frequently results from infection, trauma, drug overdose (e.g. acetaminophen) and poison ingestion (Schiødt F V, Rochling F A, Casey D L, Lee W M : Acetaminophen toxicity in an urban county hospital. N Engl J Med. 1997;337:1112-1117; Schiødt F V, Atillasoy E, Shakil A O, et al.: Etiology and outcome for 295 patients with acute liver failure in the United States. Liver Transpl Surg. 1999;5:29-34). The combination of jaundice and altered mental status is the hallmark of ALF. Other clinical signs are coagulopathy, metabolic changes and renal insufficiency or failure. Decreased liver cell mass and the rate of hepatocyte regeneration account for altered liver dullness, and determine prognosis (Wedon J. A., Harrison P. M., Keays R., Williams R., Cerebral blood flow and metabolism in fulminant liver failure. Hepatology 1994; 19: 1407-141319; Williams R. New directions in acute liver failure. J Royal College of Physicians of London 1994; 28:552-559).

The onset and severity of encephalopathy is variable. Encephalopathy is usually graded 0-4. Admission to an ICU is mandatory if higher grades develop, because patients are at risk of cerebral and multi-organ complications and mortality increases. However, rapidly worsening encephalopathy has a better prognosis than the slowly progressive form (Mullen K D, Dasarathy S. Hepatic encephalopathy. E R Schiff, M F Sorrell, W C Maddrey (eds), Schiff's Diseases of the Liver, 8^{th} edition, Lippincott-Raven Publishers, Philadelphia, 1999).

Cerebral edema is frequent in severe encephalopathy and accounts for most deaths. The specific pathogenesis is still debated; a combination of vasogenic and cytotoxic mechanisms are probably involved, with abnormalities in the blood-brain barrier, plasma and cerebral accumulation of ammonia, false neuro-transmitters, and GABA. The progression from the initial hyperkinetic, delirious and agitated state to pupil dilatation, hyperventilation and progressively increasing muscle tone indicates severe intracranial hypertension, requiring immediate intervention to avoid cerebral ischemia and herniation. Monitoring intracranial pressure is valuable but hazardous, and recording of sensory evoked potentials is valuable, at least in those patients who are eligible for transplantation.

Loss of synthesis of clotting factors and their inhibitors is primarily responsible for the markedly impaired coagulation, made worse by increased peripheral consumption. Sensitive techniques of coagulation monitoring also show a low-grade intravascular activation of coagulation. Deficiency of clotting factors and reduced platelet count and function increase the risk of bleeding. The main sites of hemorrhage include the gastrointestinal tract, usually due to stress erosions, nasopharynx and lungs, retroperitoneally, kidneys, and puncture sites.

Derangements such as hypoglycaemia, caused by hyperinsulinaemia and impaired gluconeogenesis, and electrolyte imbalances, are common. Hypokalaemia is usually related to respiratory alkalosis, whereas hyponatraemia results from water retention and an intracellular sodium shift. Metabolic acidosis occurs later, from increased serum lactate levels and decreased clearance of metabolic products.

Renal impairment occurs in up to 50% of all cases, from either direct renal toxicity or reduced intravascular filling pressures secondary to general vasodilatation and hypovolemia. It may occasionally be out of proportion to the hepatic injury, particularly where renal failure is not functional, but caused by acute tubular necrosis. Care is needed with potentially nephrotoxic agents, such as aminoglycosides, mannitol or contrast media.

Profound hemodynamic changes occur, particularly a marked reduction in systemic vascular resistance, with loss of vessel tone, and an increased cardiac output. Despite increased circulation and oxygen supply, underlying covert tissue hypoxia has been suggested. Hypotension and poor capillary perfusion because of platelet activation and white cell accumulation, followed by a reflex luxury perfusion of the remaining capillaries, may lead to insufficient oxygen extraction, anaerobic metabolism, and increased lactate levels. Recent studies also suggested pathological oxygen supply dependency, although this is controversial. As in septic shock there is massive release of nitric oxide, derived from vascular endothelial cells and from smooth muscle cells by endotoxin-induced nitric oxide synthase. This release probably leads to the circulatory disturbances found in ALF, and maintains the mechanism of tissue damage.

Pulmonary complications are not infrequent. Important causes include aspiration of gastric contents during encephalopathy, pulmonary infections, or other poorly understood conditions resulting in low pulmonary vascular resistance, increased alveolar-arterial oxygen difference or atelectasis and edema. This contributes to the development of features of the multiple organ failure syndrome including the development of the respiratory distress syndrome. Early intubation and ventilatory support can cause underestimation of the severity of pulmonary dysfunction.

Patients suffering from ALF are prone to infections, due to a loss of a wide range in host defenses, and the high frequency of bacterial and fungal infections is the second most frequent cause of death (sepsis) (Peltekian K M, Levy G A: Role of cytokines and immune mechanisms in acute liver failure. In Acute Liver Failure. Edited by Lee W M, Williams R. Cambridge: Cambridge Press; 1997). Impaired leucocyte migration, opsonisation and intracellular killing contribute to the substantial risk of sepsis. The bacterial toxins generated by the infecting organisms trigger complex immunologic reactions: A large number of mediators, including tumor necrosis factor, leukotrienes, lipoxygenase, histamine, bradykinin, serotonin, and interleukin-2, have been implicated in addition to endotoxin (the lipid fraction of the lipopolysaccharides released from the cell wall of gram-negative enteric bacilli). Presently, the only recommended therapeutic approach remains close microbiological surveillance. Prophylactic antibiotics and enteral decontamination have only a minor role: they may have an adverse effect by the selection of multiple resistant strains. (Ronco C, Brendolan A, Lonnemann G, Bellomo R, Piccinni P, Digito A, Dan M, Irone M, La Greca G, Inguaggiato P, Maggiore U, De Nitti C, Wratten M L, Ricci Z, Tetta C. A pilot study of coupled plasma filtration with adsorption in septic shock. Crit Care Med 2002;30:1250-55).

Treatment of acute liver failure has had discouraging results; survival in high-grade encephalopathy has been especially poor. This has changed with the introduction of orthotopic liver transplantation (OLT) as a practical procedure, with a constant high rate of survival in ALF patients somewhere in the range of 60-80% in 1-year survival in most centers. This option for ALF, has shifted the main objective to the assessment of prognostic factors and away from therapy in those patients, whose liver may have regenerative capability and may recover. Methods such as ‘bridging’ with auxiliary heterotopic liver transplantation, artificial liver support devices, and hepatocyte transplantation are far from clinically routine, but continue to be explored (Sussman N. L., Chong M. G., Koussayer T. et al. Reversal of fulminant hepatic failure using an extracorporeal liver assist device. Hepatology 1992; 16: 60-65; Sudan D L, Shaw B W jr. Fox I J, Langnas A N. Long-term follow up of auxiliary orthotopic liver transplantation for the treatment of fulminant hepatic failure. Surgery, 1997; 122(4):777-778; Nakae H, Yonekawa C, Wada H, Asanuma Y, Sato T, Tanaka H. Effectiveness of combining plasma exchange and continuous hemodiafiltration (combined modality therapy in a parallel circuit) in the treatment of patients with acute hepatic failure. Ther Apher. 2001;5:471-475). Medical treatment of ALF remains still mainly supportive, and resembles that for critically ill patients including mechanical ventilation and aggressive daily microbiological surveillance till recovery occurs, or OLT is considered. Unfortunately these patients often do not show clinical signs of infection, such as fever and leucocytosis.

Recently, a new treatment option has emerged for treating ALF, acute-on-chronic liver failure, end stage chronic liver disease and most importantly sepsis. This new treatment option uses extracorporeal devices having biocompatible absorbent materials that remove toxins form patient's plasma. For example, U.S. Pat. No. 6,372,482 B1 (hereinafter “the '482 patent”) issued Apr. 16, 2002 to Mitrani discloses a device for performing a biological modification of a fluid. The device in the '482 patent utilizes a series of micro-organ cultures wherein living cells such as hepatocytes are cultured in containers integrated into an extracorporeal circuit. Blood for a patent is circulated through the extracorporeal circuit where the living cells simulate the effect of the natural organ and detoxify the blood ex vivo.

Similarly, U.S. Pat. No. 6,008,049 (hereinafter “the '049 patent”) issued Dec. 28, 1999 to Naughton, et al. discloses a tissue engineering bioreactor where organ cells, such as liver cells, are cultured. The bioreactor of the '049 patent containing the cultured cells is then integrated into an extracorporeal circuit and used to assist a diseased organ to perform its intended biological function.

However, the artificial organ devices disclosed in the '482 and '049 patents rely on living cells to perform the complex functions of a living organ. The human liver is a complex organ that has not been successfully duplicated ex vivo. Moreover, these prior art devices do not provide methods for removing hog levels of cyokines, chemokines and other toxic compounds found in sever acute stage liver failure. The benefits, if any associated with these prior art devices, is thus limited to more chronic, less acute situations.

Extracorporeal circuits are well know in the prior art. However, the known extracorporeal circuits are used primarily as artificial kidneys and perfusion devices. Perfusion devices, sometime referred to as extracorporeal membrane oxygenation (ECMO) devices, are primarily used to provide circulatory assistance after open heart surgery. Kidney diafiltration, dialysis and pure hemofiltration are processes used to replace failing are diseased kidney. These devices principally rely on semi-permeable membrane technology and the principles of osmotic diffusion to remove proteins, salts and urea from the blood. Additionally, kidney dialysis can be combined with ultrafiltration to remove excess fluid from the blood or be combined with substitution infusion fluid to replace fluids and salts lost in the hemodiafiltration process. However, extracorporeal circuits used to augment and/or replace diseased kidneys are not designed to remove the complex biological toxin the liver is responsible for.

In U.S. Pat. No. 6,186,146 B1 (hereinafter “the '146 patent”) issued Feb. 13, 2001 to Glickman discloses an extracorporeal circuit having a filter device incorporated therein. Specifically, the '146 patent describes a treatment for cancer where cytotoxic drugs and biological agents are infused directly into a diseased organ. The patent's blood leaving the treated organ is diverted via an extracorporeal circuit where the cytotoxic agent and/or biological removed from the blood via an inline filter before reaching the general circulation. No details as to the filter's composition are provided. However, the simple extracorporeal circuit disclosed in the '146 patent is intended to remove a defined concentration of a specific known chemotherapeutic and/or biological. It is not intended as a general replacement for a diseased organ. Moreover, no details are provided as to how one of ordinary skill in the art would use the disclosed device to remove other biological toxins.

The scientific literature provides some interesting experimental alternatives for treating ALF and sepsis. See for example J. A. Kellum and M. K. Dishart. Effect of Hemofiltration Filter Adsorption on Circulating IL-6 Levels in Spectic Rats. Critical Care 2002, 6:429-433 (herein after “Kellum). Kellum discloses using a hydrogel-type membrane made from an acrylonitrile and sodium methallyl sulfonate copolymer to remove IL-6 from the blood of septic rats. Reduction in over all IL-6 levels were noted; however, the filter used has a limited absorption profile and not all sepsis-associated cytokines are removed.

However, many cytokines and other toxins are bound to the blood protein albumin. Conventional dialysis membranes do not remove substantial quantities of these protein-toxins form the blood because protein-impermiable membranes are generally used. Consequently, other extracorporeal circuits such as continuous renal replacement therapies (CRRT), coupled plasma filtration adsorption (CPFA) and continuous veno-venous hemodiafiltration (CWHDF) may help minimize cell-associated cytokine concentrations is the blood of septic patents. See for example C. Tetta et al. Endotoxin and Cytokine Removal in Sepsis. Ther. Apher 2002. Vol 6: No. 2 109-115. (herein after Tetta). Tetta concluded that CPFA may be preferable to CRRT and CWHDF for treating septic patents, but that much clinical research was need to prove efficacy. These more invasive detoxification methods enable higher clearance of protein bound toxins due to direct contact between the sorbent and the albumin/toxin-complex. However, negative side effects arising from prolonged plasma-sorbent contact has limited the use of these techniques (T. M. Rahman and H. J. Hodgson. Review article: Liver Support Systems in Acute Hepatic Failure. Aliment. Phamacol. Ther. 1999; 13: 1255-1272; S. R. Ash. Treatment of Acute Hepatic Failure with Encephalopathy: A review. Int. J. Artif Organs 1991; 14: 191-195.)

Consequently, there remains a need for extracorporeal devices and methods that can be used to safely remove toxins from patents suffering from ALF. More specifically, there remains a recognized need for extracorporeal devices and methods useful for treating ALF-associated sepsis.

### SUMMARY OF THE INVENTION

The present invention provides a plasma toxin removal system useful for treating patients suffering from both acute and chronic forms of liver disease. In one embodiment the present invention is a compact, mobile, self-contained device designed for use at a patient's hospital bedside where it will remove toxins from a patient's bloodstream. The present invention includes treatment kits assembled in a sterile package comprising at least one toxin removal device, a plasma separator, tubing, and a variety of other components. During a typical patient treatment, the treatment kits of the present invention are attached to roller pumps and used in association with commercially available hardware and software approved for extracorporeal applications. For example and not intended as a limitation, the commercially available B|BRAUN DIAPACT™ CRRT machine is used.

In one embodiment of the present invention a method for removing toxins from the plasma of a patient in need thereof is provided. The toxin removal process of the present invention begins with placing of a dual lumen catheter into a large vein to provide access to the patient's circulatory system thus creating an extracorporeal circuit. Next a standard roller-pump system, similar to those used in hemodialysis, is attached to the catheter. Prior to blood being allowed to enter the extracorporeal circuit physiological saline is added to the toxin removal device so that the toxin adsorbents will form a suspension that will facilitate the removal of the targeted toxins. Blood is drawn from the patient with the roller pump and passed through the plasma separator. The plasma is directed to flow through the toxin removal device of the present invention and the toxins bind to the toxin adsorbents thus removing the toxins from the plasma.

In one embodiment of the present invention an extracorporeal circuit is provided having at least toxin removal device disposed down stream of a plasma separator. The toxin removal devices of the present invention contains one or more biocompatible toxin adsorbent and/or absorbent material (referred to herein collectively as toxin removal material) that remove biological toxins, soluble proteins including cytokines and chemokines, carbohydrates, lipids, nucleic acids, glycoproteins and other soluble substances which may cause adverse physiological conditions.

In one embodiment of the present invention the toxin removal material is uncoated activated charcoal.

In another embodiment the toxin removal material is one or more non-ionic resins.

In yet another embodiment of the present invention at least one non-ionic resin is connected to the extracorporeal circuit downstream of the plasma separator and in series with the activated charcoal.

In still another embodiment of the present invention at least one non-ionic resin and the activated charcoal are combined into a single chamber or cartridge and connected to the extracorporeal circuit downstream of the plasma separator. In this embodiment the non-ionic resin(s) and charcoal maybe physically separated or mix.

One particular system embodying the present invention may comprises an ultrafiltration device placed in the blood return line of the extracorporeal circuit to facilitate excess fluid removal; a membrane oxygenator device placed in the blood return line to provide oxygen and remove carbon dioxide; a heat exchanger in the blood return line for temperature control, at least one ionic resin as directed by the physician and a heparin pump.

Still other embodiments of the present invention include one or more dead-end filters between the output side of the toxin removal device(s) and the return line to the patent. The dead-end filter retains microscopic particles that may elute from the toxin removal system with the plasma. The plasma passes through the dead-end filter before being returned to the patent.

### BRIEF DESCRIPTION OF THE FIGURES

FIG. 1 is a schematic diagram showing one embodiment of the system of the present invention having a single toxin removal device.

FIG. 2 is a schematic diagram showing another embodiment of the system of the present invention having a plurality of toxin removal devices in series.

FIG. 3 is a schematic diagram showing another embodiment of the system of the present invention having a plurality of toxin removal devices in series.

FIG. 4 is a schematic diagram showing another embodiment of the system of the present invention having a plurality of toxin removal devices in series.

FIG. 5 a-c graphically depicts the toxin removal device of the present invention's efficacy in decreasing initial blood levels of bilirubin, urea nitrogen, and creatinine.

FIG. 6 graphically depicts the toxin removal device of the present invention's efficacy in decreasing blood acetaminophen concentration.

FIG. 7 a-f graphically depicts testing demonstrating that inclusion of the present invention into an extracorporeal circuit did not result in evidence of hemodynamic instability, hemolysis, thrombocytopenia, leukopenia, or nonspecific loss of fibrinogen or albumin.

FIG. 8 depicts one embodiment of the toxin removal device made in accordance with the teachings of the present invention.

### DEFINITION OF TERMS

The following definition of terms is provided as a helpful reference for the reader. The terms used in this patent have specific meanings as they related to the invention's function. Every effort has been made to use terms according to their ordinary and common meaning. However, where a discrepancy exists between the common ordinary meaning and the following definitions, these definitions supercede common usage.

“Absorbent:” As used herein an absorbent is a medium such as activated carbon or a non-ionic resin that retains a biologically active organic molecule or inorganic salt. Generally, absorbent refers to something that absorbs. Absorption is the taking in by chemical or molecular attraction similar to how water is taken in and held by a sponge.

“Adsorbent:” A used herein adsorbent a medium such as activated carbon or a non-ionic resin that retains a biologically active organic molecule or inorganic salt. Generally, adsorbent refers to something that adsorbs. Adsorption is the taking up and holding be chemical attraction to the surface of a solid substance similar to how a cloth may adsorb large dye molecules by holding them on the surface of the fibers by chemical attraction.

“Exchange Resin” as used herein generally refers to the non-ionic exchange resin component of the present invention. Furthermore it is understood that term exchange resin may be used collectively to refer to both ion exchange resins and non-ion exchange resins in those embodiments where a ion exchange resin is added to the extracorporeal circuit in combination with the toxin removal device of the present invention.

“Toxin removal device:” as used here refers to one or more cartridges or containers that contain one or more adsorbents or absorbents capable of removing organic molecules and/or inorganic salts from plasma or other biological fluids. The inventors believe that most non-ionic resins and activated charcoal act as adsorbents by attacking to their surface and retaining thereon organic molecules and inorganic salts. However, the present inventors do not wish to be bound by this theory. Therefore, for the purposes of this invention, the term “toxin removal device” will include materials that either adsorb are absorb toxins from the blood and/or plasma of patients suffering from liver disease.

Moreover, the term “toxin removal device” can mean a single unitary device wherein one or more toxin removing compounds are contained therein, either mixed or physically separated. However, the term “toxin removal device” can also refer to a plurality of discrete unitary devices each containing one or more separate toxin removal compositions. The discrete devices may be connected in series depending on the device design and application.

“Toxin” as used herein may include any organic or inorganic compound that when present in a patient's blood above a tolerable threshold causes an adverse effect on the patent. Representative, examples include, but are not limited to cytokines including interleukins, interferons, tumor necrosis factor alpha, or gamma, soluble proteins, albumin, bilirubin, hemoglobin, amino acids, nucleic acids, bacterial toxins including endotoxins, exotoxinins, lipopolysacccharides, cellular enzymes, bacterial cell wall components and pharmaceuticals such as acetaminophen.

### DETAILED DESCRIPTION OF THE INVENTION

The present invention is directed at removing toxins form the blood that accumulate during end stage chronic liver disease and or during acute liver failure (ALF). The mammalian liver is responsible for many essential biological functions including detoxifying plasma. When the liver fails, toxins can quickly accumulate in the plasma leading to multi-organ failure, coma and eventually death. Recent efforts to using existing hemofiltration, diafiltration and diahemofiltration have only been marginally effective. One short coming of the existing extracorporeal systems is the inability of many filters to remove albumin-bound toxins. When filters useful for removing albumin-bound toxins are substituted for conventional filters, biocompatibility problem have been reported. Consequently, there remains a need for an extracorporeal system that can quickly and effectively detoxify human plasma in patients suffering from ALF and end-stage chronic liver disease.

Sepsis is a life threatening complication associated with end-stage liver disease and ALF. While the exact anatomical and physiological parameters associated with sepsis are not entirely understood, it is generally believed that sepsis is caused by the loss of the liver's structural integrity that allows normal intestinal flora to invade the blood. Furthermore, sepsis is also associated with abdominal surgery of sever burns.

The major danger associated with sepsis is septic shock caused by the release of endotoxins associated with bacterial cell walls. These toxins cause inflammatory responses by over-exciting the immune system. The immune response deals well with relatively minor invasions but, with such a massive overload, can cause major shock in which the blood pressure falls dramatically which can exacerbate liver failure. However, once sepsis has set in, treatments which kill the bacteria make the problem worse by causing the release of more bacterial endotoxins from the dying bacteria.

Therefore, whether ALF is the primary cause of sepsis, or secondary thereto, sepsis is a life threatening condition that until know was extremely difficult to treat or control. Consequently, the present inventors have developed an extracorporeal circuit useful for removing toxins from the plasma of patients suffering form ALF and or/ sepsis.

The present inventors have designed a system for detoxifying the plasma of patents in need thereof that obviates problems associated with biocompatibility, electrolyte imbalances and protein permeability associated with conventional hemofiltration/diafiltration systems. Thus, the present invention provides a blood toxin removal system that does not result in clinically significant electrolyte imbalances or excessive protein removal from the treated patients plasma. However, the present inventors have retained the simplicity and clinical acceptability of the standard extracorporeal circuits commonly used for treating kidney failure and cardio-pulmonary reperfusion. Thus it is possible to introduce the detoxifying extra corporal circuit of the present invention directly into conventional systems for continuous renal replacement therapy such as the B. Braun the Diapact™ CRRT (see http://www.bbraun.com/ for details).

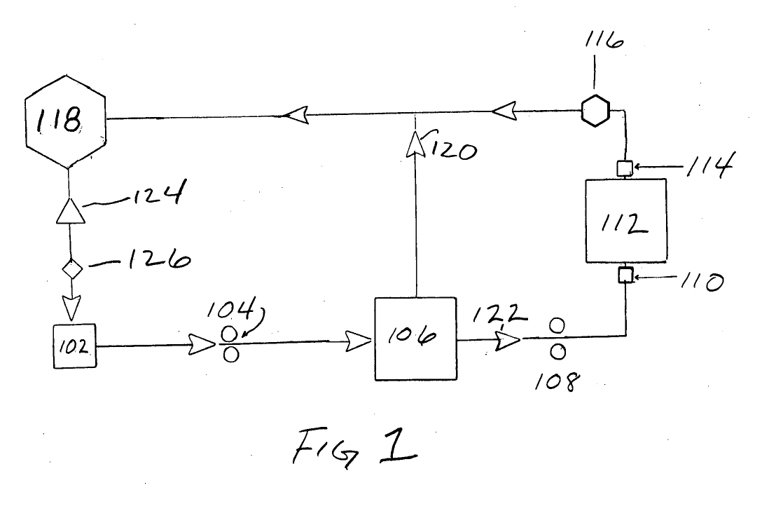

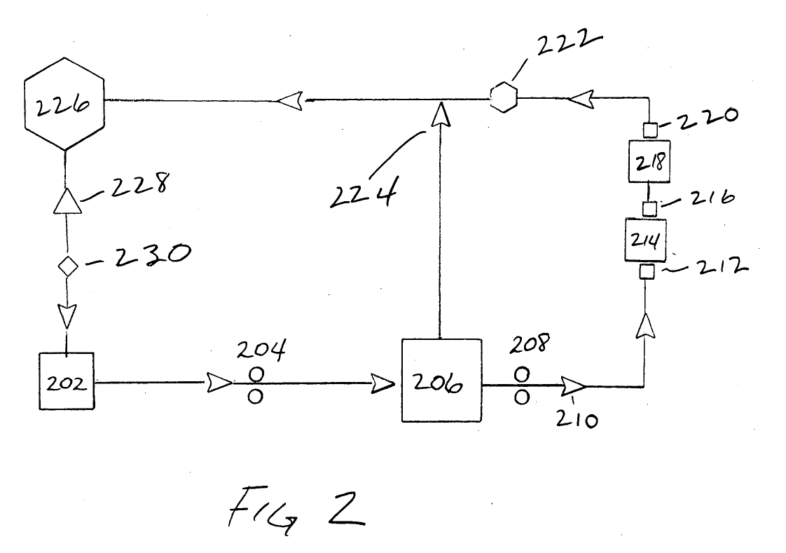

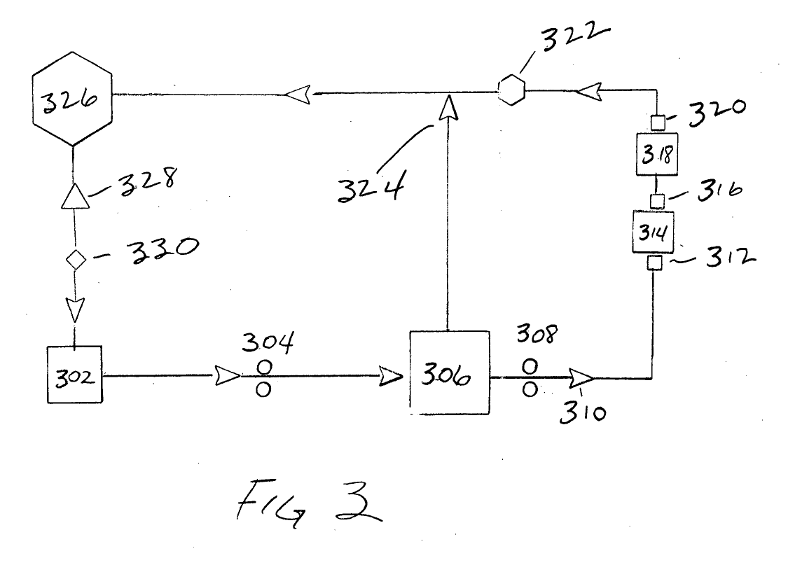

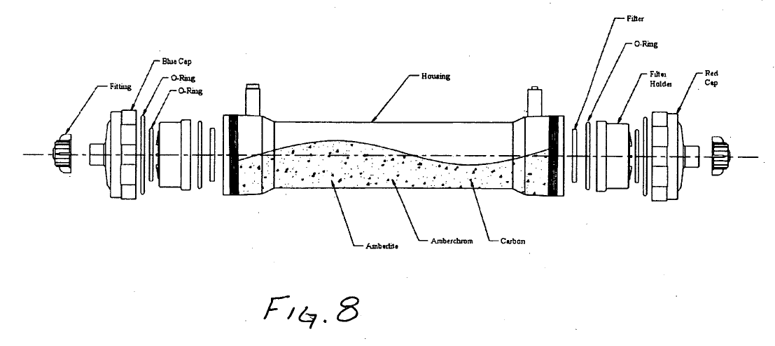

Turning now to Figures the present invention will be described generally with reference to FIG. 1 and FIG. 2. One embodiment of the present invention is depicted in FIG. 1. In FIG. 1 blood is aspirated from a patient 102 in need of plasma detoxification using a conventional dual lumen renal catheter connected to a peristaltic pump 104 and directed into a plasma filter 106 where the blood cells are separated from the plasma fraction of the blood. In one embodiment of the present invention a suitable plasma separation filter is a hollow fiber filter having a total surface are of 1 meter and a 0.45 μM cutoff is provided by Minntech, Inc. (Minneapolis, Minn. 55447). The separated blood leaves the hollow fiber plasma filter 106 by one of two routes. Blood cells are returned to the patent via pathway 120 and the separated plasma enters pathway 122. At pathway 122 the separated plasma moves into the toxin removal device of the present invention 112 (see also FIG. 8). The separated plasma may be assisted by optional pump 108 and may be optionally pre-filtered through pre-filter 110 prior to entering the toxin removal device 112. Next the detoxified plasma exits the toxin removal device 112 and optionally passes through a second pre-filter 114 before entering the dead-end filter 116. Pre-filters 110 and 114 may be composed of any one of different compounds including, but not limited to polypropoylene and generally have a pore size in the range of approximately 3 μM to 5 μM. The dead-end filter 116 may be composed of any biocompatible material and generally has a pore size that does not exceed 0.45 μM. The dead-end filter assures that and micro-particulates released of the upstream devices is removed from the detoxified plasma before being returned to the patient.

The detoxified plasma is returned to patient via pathway 120 where it rejoins the separated blood cells and together are pumped through a hemoconcentration filter 118 that removes excess fluid from the blood. The returning blood may then optionally be heated by heater 124 an then passes into bubble trap 126 before returning the patient 102.

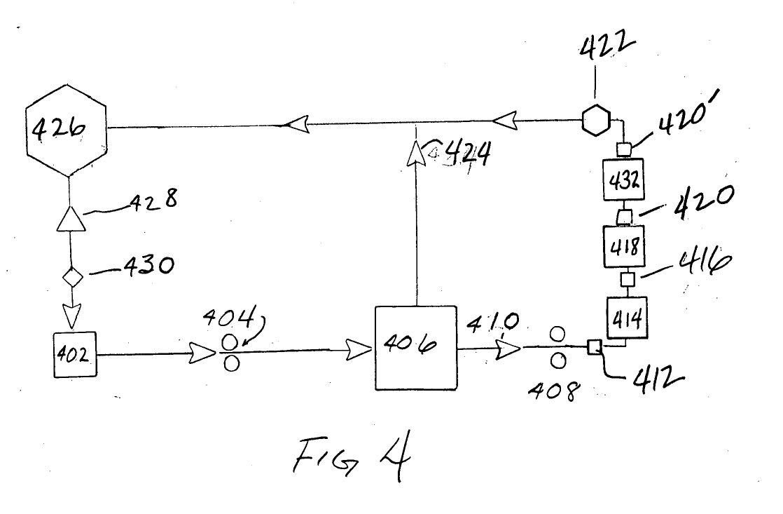

FIG. 2 depicts another method for practicing the present invention. In FIG. 2, as in FIG. 1 the process begins as blood is aspirated from a patient 202 in need of plasma detoxification using a conventional dual lumen renal catheter connected to a peristaltic pump 204 and directed into a plasma filter 206 where the blood cells are separated from the plasma fraction of the blood. In one embodiment of the present invention a suitable plasma separation filter is a hollow fiber filter having a total surface are of 1 meter and a 0.45 μM cutoff is provided by Minntech, Inc. (Minneapolis, Minn. 55447). The separated blood leaves the hollow fiber plasma filter 206 by one of two routes. Blood cells are returned to the patient via pathway 224 and the separated plasma enters pathway 210. At pathway 210 the separated plasma moves into the first toxin removal device of the present invention at 214. The separated plasma may be assisted by optional pump 208 and may be optionally pre-filtered through pre-filter 212 prior to entering the first toxin removal device 214. Next the partially detoxified plasma exits the first toxin removal device 214 and optionally passes through a second pre-filter 216 before entering a second toxin removal device 218. After passing through the second toxin removal device 218, the detoxified plasma may then optionally pass through a third pre-filter 220 before entering the dead-end filter 222. Pre-filters 212 (and 420′ in FIG. 4), 216 and 220 may be composed of any one of different compounds including, but not limited to polypropoylene and generally have a pore size in the range of approximately 3 μM to 5 μM. The dead-end filter 222 may be composed of any biocompatible material and generally has a pore size that does not exceed 0.45 μM. The dead-end filter assures that and micro-particulates released of the upstream devices is removed from the detoxified plasma before being returned to the patient.

The detoxified plasma is returned to patient via pathway 224 where it rejoins the separated blood cells and together are pumped through a hemoconcentration filter 226 that removes excess fluid from the blood. The returning blood may then optionally be heated by heater 228 an then passes into bubble trap 230 before returning the patient 202.

When multiple toxin removal devices are used as depicted in FIG. 2 it is not important which type of toxin removal device the plasma enters first. Moreover, the present inventors envision embodiments where the more than two toxin removal devices are attached in series, that is a plurality of toxin removal devices wherein a plurality denotes two or more such devices. In one embodiment of the present invention the first toxin removal device 214 is activated charcoal and the second toxin removal device 218 is non-ionic resin. In another embodiment of the present invention the first toxin removal device 214 is non-ionic resin and the second toxin removal device 218 is charcoal. In yet still another embodiment both toxin removal devices are the same and may contain both activated charcoal and/or non-ionic resins.

Furthermore, it is understood that substitution infusion fluids such as this used in renal dialysis may be added to the extracorporeal circuit of the present invention at one ore more paces in the process. For example, and not intended as a limitation substitution infusion fluid may be added before the blood reaches the plasma filter 106 or 206. In another embodiment the substitution infusion fluid may be added before entering the hemoconcentration filter 118, 226 or at any point in between these two points in either circuit 120/224 or 122/210.

The toxin removal devices of the present invention comprise biologically active materials that adsorb (or absorb see discussion supra) blood borne toxins that accumulate due to diminished liver function. The toxin removal devices of the present invention may contain one or more material selected from the group consisting of activated charcoal and ion exchange resins. Essentially, ion exchange resins are classified as cation exchangers, which have positively charged mobile ions available for exchange, anion exchangers, whose exchangeable ions are negatively charged and non-ionic exchange resins that bind macromolecules via intermolecular forces, also referred to as van der Waal's forces, the weak attractive forces that hold non-polar molecules together (or non-polar regions of molecules having polar groups).

Both anion and cation resins are produced from the same basic organic polymers. However, they differ in the ionizable group attached to the hydrocarbon network. It is this functional group that determines the chemical behavior of the resin. Ionic exchange resins can be broadly classified as strong or weak acid cation exchangers or strong or weak base anion exchangers. In an ion exchange process, cations or anions in a liquid solution (usually aqueous) replace dissimilar and displaceable ions of the same charge contained in the ion exchange resin.

Non-ion exchange resins are particular advantageous when used in accordance with the teachings of the present invention because they are less prone to bind (and thus removed from the blood) essential actions and anions such as, but not limited to calcium, magnesium, sodium, potassium, chloride, carbonates, and other ionic species. Consequently, it is not necessary to carefully monitor, as required, balance electrolyte concentrations in the patient's blood during prolonged treatment. However, as previously discussed, it is still possible to replenish electrolytes as needed at the physician's discretion by combining the present invention with conventional substitution and infusions fluids as known to those have ordinary skill in the art of physiology.

Specific non-limiting examples of non-ionic exchange resins suitable for use with the present invention include Amberlite™ XAD-7 HP and Amberchrom™ CG300-C. Amberlite™ is a group of polymeric synthetic resins made by the Rohm and Haas Company having a North American headquarters at 100 Independence Mall West Philadelphia, Pa. 19106-2399. Amberlite™ resins are available world wide thorough a distributor network know to those skilled in the art. In one specific embodiment the present inventors have used Amberlite™ XAD-7 HP which is an aliphatic ester resin having an average surface area of approximately 500 m²/g and an average pore size of approximately 450 Angstroms and a mean diameter of 560 μm.

In another embodiment of the present invention the inventors have used Amberchrome™ CG300-G. Amberchrome™ is also made by Rohm Haas and is available world-wide. Amberchrome™ CG300-G is a synthetic ion exchange resin made from polystyrene divinyl benzene having an average surface area of approximately 700 m²/g with an average pore size of 300 Angstroms; mean particle diameter ranges from approximately 35 μM to approximately 120 μM.

However, whether the non-ion exchange resins are used individually or in combination is not meant to be limiting, persons having ordinary skill in the art can easily select the exchange resin(s) best suited for a particular application. The factors that should be considered when selecting an appropriate exchange resin include the size, shape and charge of the molecule. Toxic peptides are general small and possess a few areas of high electron density but are known to possess carboxylic acid and amine residues that are easily polarizable and capable of hydrogen bonding. Larger macromolecules including cytokines, lymphokines and other toxic proteins have strong intermolecular forces suitable for removal using non-ionic resins that depend on van de wall forces to attract and bind molecules.

The activated carbon component of the toxin removal device of the present invention comprises elementary carbon in a graphite like structure. It can be produced by heat treatment, or “activation,” of raw materials such as wood, coal, peat and coconuts. During the activation process, the unique internal pore structure is created, and it is this pore structure which provides activated carbon its outstanding adsorptive properties. Activated carbon is a carbonaceous adsorbent with a high internal porosity, and hence a large internal surface area. Commercial activated carbon grades have an internal surface area of 500 up to 1500 m²/g. Two representative, non-limiting examples of commercially available activated carbon include Carbomix™, available from Norit, Nederland B.V. Headoffice P.O. Box 105 3800 AC Amersfoort, The Netherlands and Ultracarbon™ available through Merck & Co., Inc. Whitehouse Station, N.J.

The toxin removal devices of the present invention generally comprise a combination of at least one or more exchange resins and activated carbon. In one embodiment of the present invention a toxin removal device is a unitary structure having disposed therein at least one non-ionic resin in combination with activated charcoal. The unitary structure, such as a tubular member, may contain a homogenous mixture of the non-ionic exchange resin(s) and charcoal, or may have the charcoal and non-ionic change resin(s) separated into discrete chambers. In another embodiment of the present invention the toxin removal device may include a plurality of separate structures connected in series as depicted in FIG. 2. In yet another embodiment the toxin exchange devices may include additional structures having cationic/anionic (or combinations thereof) ion exchange resins (hereinafter “ionic” ion exchange resins) connected in series with the charcoal and non-ionic exchange resins.

FIG. 3 depicts an embodiment wherein the toxin removal device includes the additional feature of at least one ionic ion exchange resin component 318 down stream (or alternatively upstream) of the toxin removal device 314 wherein the toxin removal device 314 is a composite device having therein charcoal and at least one non-ionic resin. In an exemplary, non-limiting embodiment, the ionic ion exchange resin of 318 is anion exchange resin, in another embodiment 318 is a cation exchange resin and in yet a third embodiment 318 is a mixed bed ionic ion exchange resin (cation mixed with an anion exchange resin). In FIG. 3 numbers 302, 304, 306, 308, 310, 312, 316, 320, 322, 324, 326, 328 and 330 correspond to numbers 202, 204, 206, 208, 210, 212, 216, 220, 222, 224, 226, 228 and 230 in FIG. 2.

FIG. 4 depicts an embodiment wherein the toxin removal device includes the additional feature of at least one ionic ion exchange resin component 432 down stream (or alternatively upstream) of a toxin removal devices connected in series 414 and 418 wherein the toxin removal devices 414 and 418 are charcoal and at least one non-ionic resin respectively (or visa versa). In an exemplary, non-limiting embodiment, the ionic ion exchange resin of 432 is anion exchange resin, in another embodiment 432 is a cation exchange resin and in yet a third embodiment 432 is a mixed-bed ionic ion exchange resin (cation mixed with an anion exchange resin). Reference number 434 is a pre-filters composed of any one of different compounds including, but not limited to polypropoylene and generally have a pore size in the range of approximately 3 μM to 5 μM. In FIG. 4 numbers 402, 404, 406, 408, 410, 412, 416, 420, 422, 424, 426, 428 and 430 correspond to numbers 202, 204, 206, 208, 210, 212, 216, 220, 222, 224, 226, 228 and 230 in FIG. 2.

In a specific embodiment the present invention as depicted in FIG. 2 toxin removal device comprises an activated carbon column 214, non-ionic adsorption materials 218 (activated carbon, Amberlite™ XAD-7 HP resin and Amberchrom™ CG300-C), and a 3-5 micron polypropylene depth filter pad attached to support structures to entrain the adsorbent material in the column 212, 216 and 220 in conjunction with a commercially available plasma filter 206 with a 0.20-0.45 micron permeability adapted to be used in conjunction with a commercially available continuous renal replacement therapy (CRRT) machine, such as, but not limited to the BBraum Diapact™. FIG. 2 depicts a patient 202 being in fluid communication with the toxin removal device of the present invention via a CRRT machine. A particle filter 222 such as one manufactured by Minntech (FiberFlo™ Capsule Water Filter, for example) is used downstream from the toxin removal device to filter any small particles prior to return to the patient.

A standard dual lumen hemodialysis catheter is required for performing treatments. Blood is removed through the arterial line of the hemodialysis catheter by the action of the continuous roller pump 204 at a relatively low blood flow rate of approximately 125 ml/min (see FIG. 2 below). The toxin-containing blood then enters a plasma filtration step 206. The plasma filter provides the continuous plasma filtration mode to generate plasma. This ensures that low-, middle, and large molecular weight toxins are able to come into direct contact with the toxin removal device. In the next step while the cellular components of blood such as RBCs, platelets and leukocytes remain separate to avoid the drawbacks of direct hemoperfusion columns. Previous hemoperfusion columns were placed directly in the blood path, which allowed for activation and sequestration of platelets. The plasma filtrate that is generated is pumped by a second roller pump 208 at a rate of approximately 25 mL/min and passed through the toxin removal device 214 and 218 containing activated uncoated coconut shell (carbon granules) charcoal (100 gm), and the nonionic resins Amberlite XAD-7HP (30 gm) and Amberchrom GC300C (35 gm) . During the priming phase of preparation for toxin removal treatment, albumin in the priming solution coats the toxin removal device further increasing their biocompatibility.

The detoxified plasma is then rejoined to the blood path 224 and is subsequently returned to the patient 202 through the venous line of the hemodialysis catheter. A commercially available hemoconcentrator 226 [Minntech HPH 400TS™] may be added to the circuit to enable ultrafiltration fluid removal, at the discretion of the treating physician, depending on patient needs.

The safety of extracorporeal detoxification utilizing the commercially available B|BRAUN DIAPACT™ CRRT machine Plasma Adsorption/Perfusion (PAP) mode has been demonstrated. In one embodiment of the present invention the B|BRAUN DIAPACT™ CRRT machine in PAP mode is utilized in accordance with it's approved labeling including the use of standard PAP mode tubing, hardware, software and safety settings. The B|Braun Diapact™ CRRT machine in PAP mode is currently used clinically with the Asahi Medical Co. PlasmaFlo plasma filter and the Asahi CH-350 charcoal hemoperfusion column. The safety and efficacy of the substitution of the present invention for the Asahi charcoal column in an extracorporeal circuit controlled with the B|BRAUN DIAPACT™ CRRT machine in PAP mode should be demonstrated by on going clinical studies.

### EXAMPLES

The following Examples are not intended as limitations. Rather they demonstrate illustrative embodiments of the present invention.

### Example 1

### In Vitro Clearance Capabilities of the Toxin Removal Device

To demonstrate the efficacy of the toxin removal device (FIG. 8) of the present invention human plasma spiked with bilirubin (20 mg/dL), urea nitrogen (50 mg/dL), and creatinine (5 mg/dL) was circulated through a closed system with separate columns containing activated charcoal, Amberlite XAD-7HP, and Amberchrom GC 300C (referred to herein individually as “sorbants”) for 6 hours. The sorbants demonstrated varying effectiveness in clearing bilirubin, urea nitrogen and creatinine: activated charcoal (36 gm) decreased the levels of bilirubin by 49.5%, urea nitrogen 24.7%, and creatinine 97.9% of baseline values; Amberlite XAD-7HP (31 gm) decreased the levels of bilirubin by 34.6%, urea nitrogen 11.2%, and creatinine 9.0% of baseline values; Amberchrom GC300C decreased the levels of bilirubin by 95.7%, urea nitrogen 11.2%, and creatinine 10.1% of baseline values. Associated with these clearances was a modest 15-20% decrease in plasma albumin and total protein concentrations.

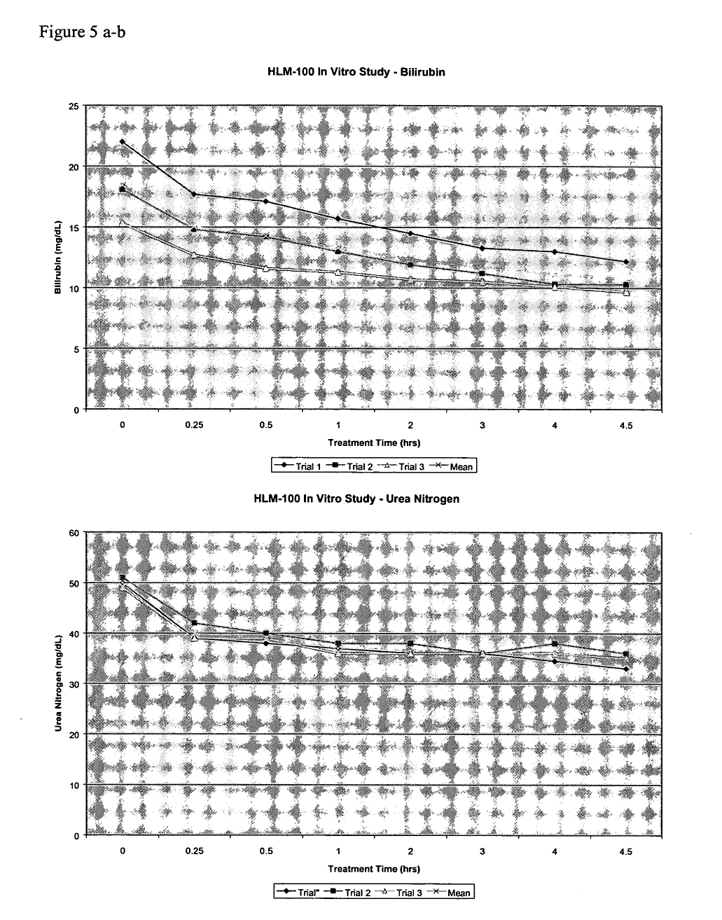

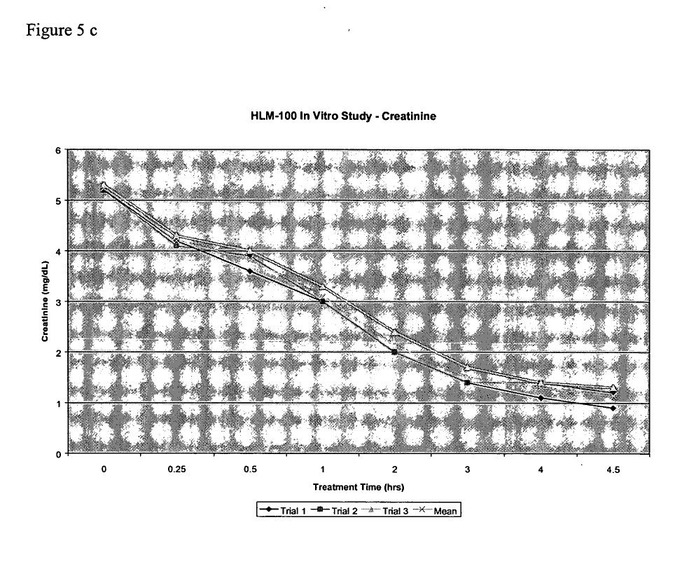

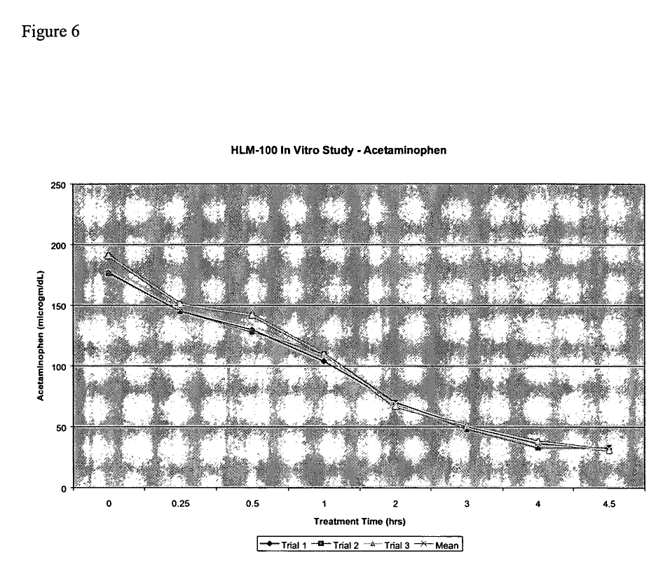

A separate series of in vitro experiments was carried out utilizing the toxin removal device containing activated 100 gm uncoated coconut shell granule charcoal, 30 gm Amberlite™ XAD-7HP, and 35 gm Amberchrom™ GC300C (dry weights). Heparinized human plasma spiked with bilirubin, urea nitrogen, and creatinine at approximate initial concentrations of 20 mg/dL, 50 mg/dL, and 5 mg/dL, respectively. The combination toxin removal device of the present invention decreased initial bilirubin, urea nitrogen, and creatinine levels by 41.4%, 30.7%, 78.3% respectively (see Table 1 and FIG. 5). In addition to these endogenous toxins, acetaminophen was added to the plasma at an initial concentration of approximately 175-200 micrograms/mL. The toxin removal device of the present invention decreased the initial acetaminophen concentration by 82.4% (see FIG. 6). There was a modest 10-15% decrease in total protein and albumin, in addition to a 25-30% fibrinogen decline.

This in vitro data confirms the ability of the toxin removal device of the present invention to effectively remove associated with acute liver failure and acute-on-chronic liver failure. The clearance of bilirubin also indicates the clearance of bile acids, both of which are toxic to hepatocytes and impair CNS function and the immune response to infection. The removal of acetaminophen confirms the present invention's ability to remove exogenous toxins in a similar manner to the two predicate devices described above.

TABLE 1
In vitro Investigation of Toxin Removal of the Present invention.

| | |
|---|---|
| | Percent Decrease in Initial Toxin Levels |
| Toxins | (Percent ± SD) |

| | |
|---|---|
| Endogenous Toxins | |
| bilirubin | 41.4 ± 4.2 |
| Urea Nitrogen | 30.7 ± 2.9 |
| creatinine | 78.3 ± 3.8 |
| Exogenous Toxin | |
| Acetaminophen | 82.4 ± 1.3 |

### Example 2

### Safety Testing of the Present Invention

The safety of the present invention was also investigated in an animal extracorporeal circulation model (canine model involving eight approximately 55 pound mongrel dogs). The test results obtained demonstrated that treatments using the present invention in conjunction with the B|BRAUN DIAPACT™ CRRT machine in PAP mode were safe and well tolerated without detrimental hemodynamic effects or biocompatibility concerns.

Testing involved canine model extracorporeal circulation with the B|BRAUN DIAPACT™ CRRT machine in PAP mode was performed for a lead in hour to determine the effects of the extracorporeal circuit without inclusion of plasma filtration and the present invention (blood loop). The plasma flow pump was then initiated with the present invention included into the plasma flow path of the extracorporeal circulation (plasma loop) for an additional four hours (total of 6 liters of plasma processed by present invention).

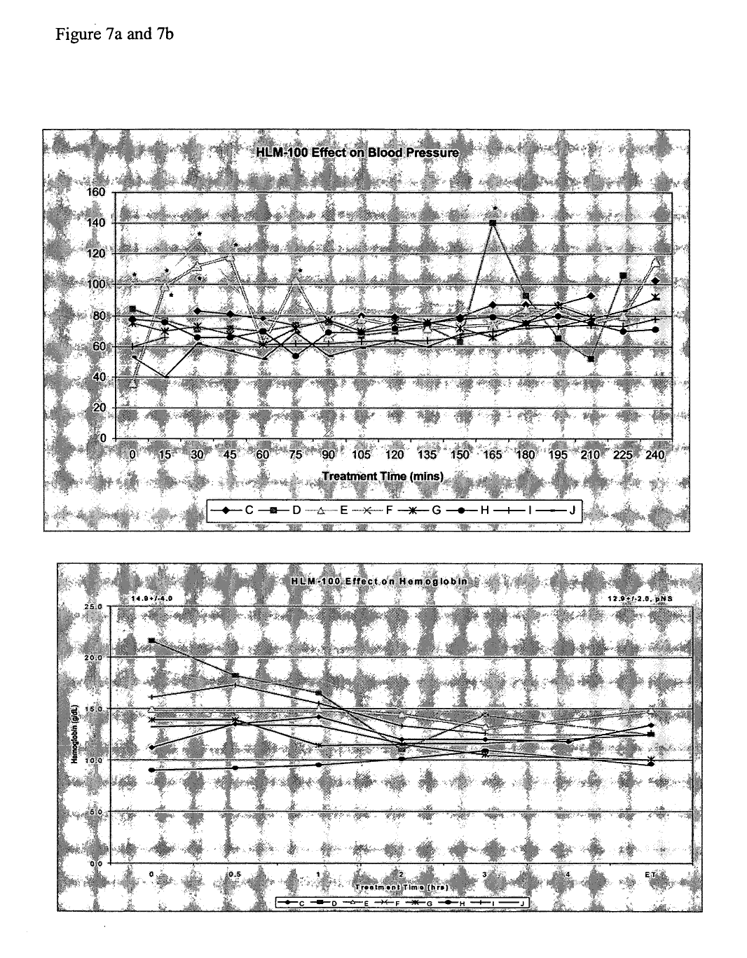

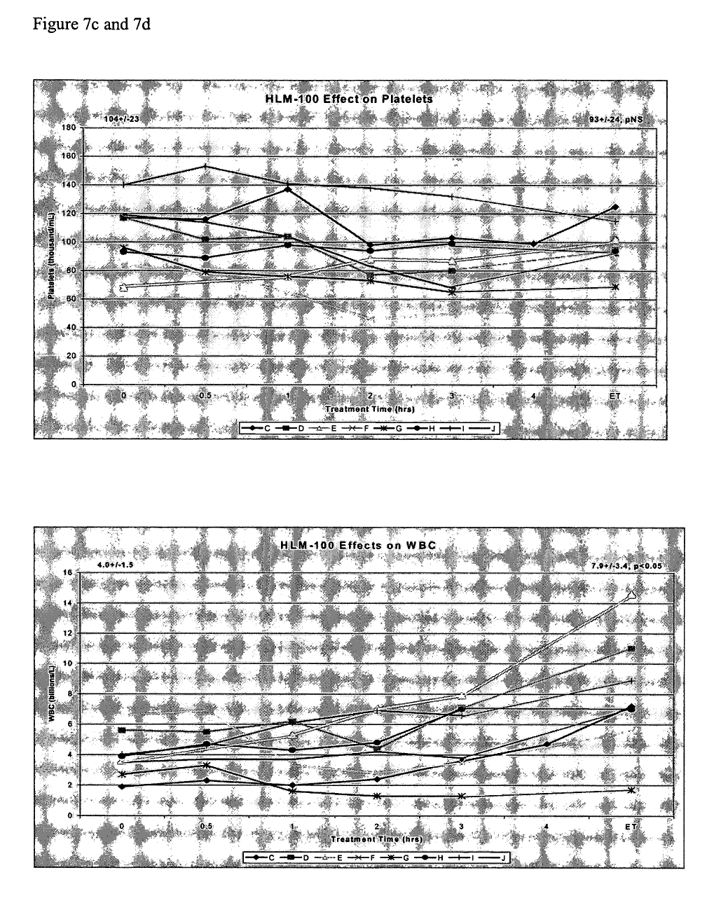

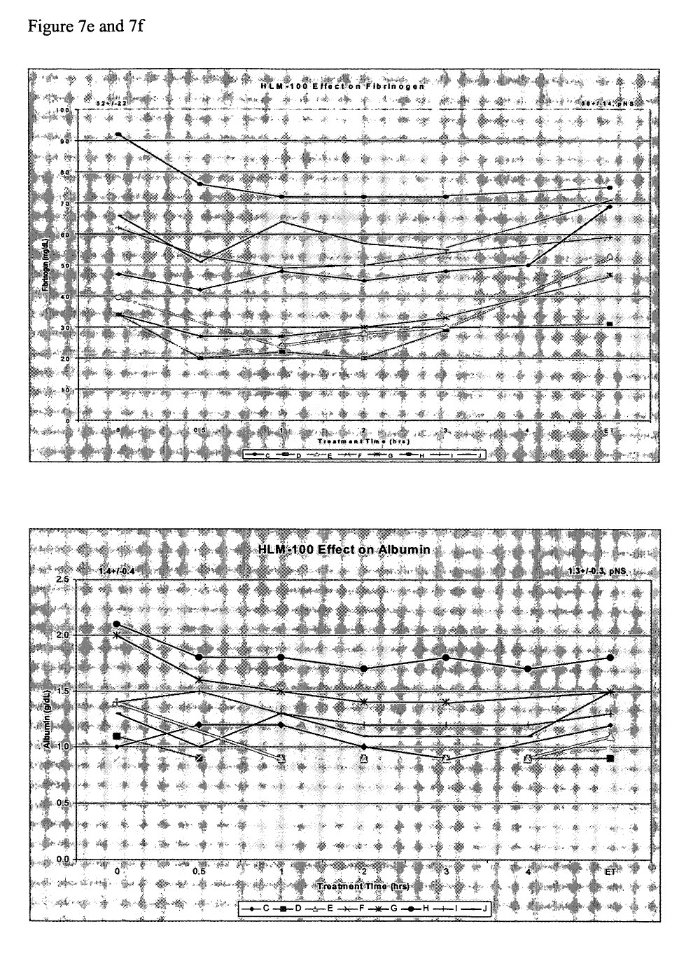

Testing demonstrated that inclusion of the present invention into an extracorporeal circuit did not result in evidence of hemodynamic instability, hemolysis, thrombocytopenia, leukopenia, or nonspecific loss of fibrinogen or albumin (see FIG. 7a-f. ET=End of Treatment values). Over the course of the 4 hours of extracorporeal circulation, including the present invention, there was an increase in mean arterial pressure. Comparison of the various parameters pre- and post-present invention inclusion confirmed that the inclusion of the adsorbent column was safe (see Table 2). There was also no evidence of present invention-related electrolyte abnormalities or consumption of clofting factors (data not shown). There was minor anticoagulation-related bleeding noted at the cut down sites for the hemodialysis catheter in addition to the invasive hemodynamic monitoring catheters (pulmonary artery catheter and arterial catheter).

TABLE 2
A comparison of the effect of the present invention inclusion into an
extracorporeal circuit in a canine model prior to and following
conclusion of 4 hours of treatment.

| | Prior to HLM- | Following HLM- | |
|---|---|---|---|
| | 100 column | 100 column | |
| | inclusion | inclusion in | p |
| | in circuit | circuit for 4 hours | value |
| Mean Arterial Pressure | 71 ± 20 | 92 ± 16 | <0.05 |
| (mmHg) | | | |
| Hemoglobin (g/dL) | 14.8 ± 4.0 | 12.6 ± 2.0 | NS |
| Platelet (Thousand/mL) | 104 ± 23 | 93 ± 24 | NS |
| WBC (Thousand/mL) | 4.0 ± 1.5 | 7.9 ± 3.8 | <0.05 |
| Serum Fibrinogen (mg/dL) | 52 ± 22 | 58 ± 14 | NS |
| Serum Albumin (g/dL) | 1.4 ± 0.4 | 1.3 ± 0.4 | NS |

In one embodiment the extracorporeal circuit including the present invention includes a self-monitoring mode that detects minor problems during a treatment and sounds an audible alarm and visual alert and thus does not require continuous input form specially trained nurses or technicians. If a major problem malfunction occurs the present invention sounds an alarm and then enters a “safe mode” wherein the system stops its operation. The treatment nurse or technician can easily resolve the alarm conditions by following the hardware system visual screen prompts. Depending on a patient's size, the entire blood volume of a patient passes through the machine every 20-30 minutes, each time removing more and more of the targeted toxins present in the blood. However, less than one unit of blood is outside of the patient at any given time adding to the overall system.

The present invention as describes and enabled herein is a toxin removal device suitable for use in an extracorporeal circuit. One useful extracorporeal circuit device is the B|BRAUN DIAPACT™ CRRT machine. The toxin removal device of the present invention comprises at least one housing, such as a cylindrical column, having activated charcoal and at least one non-ionic exchange resin disposed therein, or in individual discrete housings. The toxin removal device may also include one or more filters to prevent particulates from entering the blood of the patient undergoing treatment. One proposed trade name for the present invention's commercial embodiment is “HLM-100 Carbon Column™.” The present invention has demonstrated is suitability for removing toxins from the blood of patients suffering from acute liver failure, acute-on-chronic liver failure and sepsis. Moreover, safety has also been demonstrated in vivo.

We claim:

1. A toxin removal device for use in an extracorporeal circuit comprising:
activated charcoal and at least one non-ionic exchange resin.

2. The toxin removal device according to claim 1 where in said non-ionic exchange resin comprises a non-ionic aliphatic ester resin or a non-ionic polystyrene divinyl benzene resin.

3. The toxin removal device according to claim 2 wherein said non-ionic aliphatic ester resin has an average surface area of approximately 500 m²/g and an average pore size of approximately 450 Angstroms and a mean diameter of 560 μm.

4. The toxin removal device according to claim 3 wherein said non-ionic aliphatic ester resin is known commercially as Amberlite™ XAD-7HP.

5. The toxin removal device according to claim 4 wherein said non-ionic polystyrene divinyl benzene resin has an average surface area of approximately 700 m²/g with an average pore size of 300 Angstroms and a mean particle diameter from approximately 35 μM to approximately 120 μM.

6. The toxin removal device according to claim 5 wherein said non-ionic polystyrene divinyl benzene resin is known commercially as Amberchrom™ GC 300C.

7. The toxin removal device according to claim 1 wherein said charcoal comprises uncoated coconut shell granule charcoal.

8. The toxin removal device according to claim 1 further comprising at least one particle filter downstream of said toxin removal device in said extracorporeal circuit.

9. A toxin removal device for use in an extracorporeal circuit comprising:
activated charcoal and a non-ionic exchange resin selected from the group consisting of non-ionic aliphatic ester resin or a non-ionic polystyrene divinyl benzene resin.

10. A toxin removal device for use in an extracorporeal circuit comprising:
activated charcoal and a non-ionic exchange resin, non-ionic aliphatic ester resin and a non-ionic polystyrene divinyl benzene resin.

11. The toxin removal device according to claim 9 or 10 wherein said non-ionic aliphatic ester resin has an average surface area of approximately 500 m²/g and an average pore size of approximately 450 Angstroms and a mean diameter of 560 μm.

12. The toxin removal device according to claim 11 wherein said non-ionic aliphatic ester resin is known commercially as Amberlite™ XAD-7HP.

13. The toxin removal device according to claim 11 wherein said non-ionic polystyrene divinyl benzene resin has an average surface area of approximately 700 m²/g with an average pore size of 300 Angstroms and a mean particle diameter from approximately 35 μM to approximately 120 μM.

14. The toxin removal device according to claim 9 or 10 wherein said charcoal comprises uncoated coconut shell granule charcoal

15. An extracorporeal circuit for removing toxins from the blood comprising:
a plasma filter, an activated charcoal contained in a first housing and a second housing having at least one non-ionic resin disposed therein.

16. The extracorporeal circuit according to claim 15 wherein said at least one non-ionic resin is a non-ionic aliphatic ester resin or a non-ionic polystyrene divinyl benzene resin or a combination thereof.

17. A method for removing toxins from blood comprising:
circulating the venous blood of a patient through an extracorporeal circuit having a toxin removal device disposed therein wherein said toxin removal device comprises activated charcoal and at least one non-ionic resin.

18. The method for removing toxins from blood according to claim 17 where in said venous blood is circulated through an extracorporeal circuit wherein said at least one non-ionic resin is non-ionic aliphatic ester resin or a non-ionic polystyrene divinyl benzene resin or a combination thereof.
